(19) United States
(12) Patent Application Publication
(10) Pub. No.: US 2013/0190626 A1
(43) Pub. Date: Jul. 25, 2013

(54) DETERMINING LOCATION OF, AND IMAGING, A SUBSURFACE BOUNDARY

(71) Applicants: Andrej Bona, Shelley (AU); Roman Pevzner, Bicton (AU)

(72) Inventors: Andrej Bona, Shelley (AU); Roman Pevzner, Bicton (AU)

(73) Assignee: CURTIN UNIVERSITY OF TECHNOLOGY, Bentley (AU)

(21) Appl. No.: 13/813,639

(22) PCT Filed: Aug. 2, 2010
(86) PCT No.: PCT/AU2010/000972

§ 371 Date: Apr. 11, 2013

(51) Int. Cl. A61B 8/08; A61B 8/00; G01N 29/024

(52) U.S. Cl. CPC A61B 8/0858; G01N 29/024; A61B 8/4444; A61B 8/4477; A61B 8/5207
USPC 600/449; 367/99

(57) ABSTRACT

A method of determining a location (x,z) of a subsurface boundary (38) of a body (30), comprising providing a wave source (34) and a receiver (36) on a surface (32) of body (30); measuring a travel time of a wave from source (34) reflected to receiver (36) via the boundary (38); and determining horizontal slowness of the wave at the receiver (36) and/or the source (34), as well as the curvature of the wave at the receiver or the source. The location (x,z) is determined on the basis of the measured travel time and the determined horizontal slowness(es) and/or curvature of the wave. The wave may be a seismic wave for geophysical surveying purposes, or an ultrasound wave for medical diagnostic purposes.

### FIELD OF THE INVENTION

The present invention relates to imaging techniques that rely on reflection or diffraction of elastic waves such as, but not limited to, ultrasonic waves.

### BACKGROUND OF THE INVENTION

Imaging of the subsurface structure of a body using elastic waves is a well developed art applied in many fields of endeavour including geophysical exploration (surveying), non-destructive testing, and in medical applications. Depending on the technique used, it may be necessary to have information relating to the velocity of elastic waves through the body being imaged in order to accurately position the location of a boundary between different internal structures or media within the body and subsequently construct an image of the subsurface. For example, in seismic surveying, it is often necessary to construct or have prior knowledge of a velocity model of the subsurface in order to facilitate migration of gathers to enable imaging of the subsurface. On the other hand, in various medical imaging techniques such as in ultrasound tomography, images are produced by focusing a pulse ultrasound beam into the body and processing the reflections or echoes of the waves on an assumption of a predetermined constant velocity of the ultrasound waves through the body. However the assumption of constant velocity of ultrasound propagation in human tissue is almost always violated as velocity in soft tissue can vary by up to 15%. This variation in velocity might result in a soft focus when using current imaging methods.

The present invention was developed as a result of research into possible techniques for determining the location of a subsurface boundary without knowledge, or prior assumption of, propagation velocity of an elastic wave through a body.

### SUMMARY OF THE INVENTION

One aspect of the invention provides a method of determining a location of a subsurface boundary in a body of material comprising:
providing a first domain device and second domain device on a surface of the body where the first domain device is an elastic wave source or an elastic wave receiver, and the second domain device is the other of the elastic wave source or an elastic wave receiver;
measuring travel time of a wave propagating via a subsurface boundary between the first and second domain devices;
determining: slowness of the wave at the first domain device in a plane tangent to the surface at the first domain device; and, curvature of the wave at the first domain device; and,
calculating the location of the subsurface boundary on the basis of the measured travel time, and the determined slowness of the wave and curvature of the wave.

Determining slowness in the tangent plane may comprise determining a first derivate of travel time at the first domain device.

Determining curvature may comprise determining a second derivate of travel time at the first domain device.

In one embodiment when the first domain device is at a location (x₁,z₁) and the second domain device is at a location (x₂,z₂) and travel time of a wave from the first source to the first receiver via the boundary is measured as t, the slowness of the wave in a plane tangent to the surface at the second domain device is defined as:

$${p}_{x} = \frac{d t}{d {x}_{2}} ;$$

curvature of the wave is defined as

$${p}_{xx} = \frac{{d}^{2} t}{d {x}_{2}^{2}}$$

and
the location of a point on the boundary (x,z) impinged by the wave is determined as

$$x = \frac{\begin{matrix}2 {p}^{2} {x}_{2} ( t + \sqrt{{p}^{2} - {p}_{x}^{2}} ( {z}_{2} - {z}_{1} ) ) + \\ {p}_{x} ( {p}^{2} ( {x}_{1}^{2} - {x}_{2}^{2} + {( {z}_{2} - {z}_{1} )}^{2} ) - {t}^{2} )\end{matrix}}{2 {p}^{2} ( t + {p}_{x} ( {x}_{1} - {x}_{2} ) + \sqrt{{p}^{2} - {p}_{z}^{2}} ( {z}_{2} - {z}_{1} ) )}$$

$$and$$

$$z = \frac{\begin{matrix}\sqrt{{p}^{2} - {p}_{x}^{2}} ( {t}^{2} - {p}^{2} ( {( {x}_{2} - {x}_{1} )}^{2} + {z}_{1}^{2} - {z}_{2}^{2} ) ) + \\ 2 {p}^{2} {z}_{2} ( t - {p}_{x} ( {x}_{2} - {x}_{1} ) )\end{matrix}}{2 {p}^{2} ( t + {p}_{x} ( {x}_{1} - {x}_{2} ) + \sqrt{{p}^{2} - {p}_{x}^{2}} ( {z}_{2} - {z}_{1} ) )}$$

$$where {p}^{2} = {p}_{x}^{2} + t {p}_{xx} .$$

Providing the first and second domain devices may comprise providing the first and second domain device as an ultrasound source and an ultrasound receiver respectively.

In a second aspect the invention provides a method of determining a location of subsurface boundary in animal tissue comprising:
using the method according to the first aspect wherein the body of material is an animal body, and the first and second domain devices comprises an ultrasound source and an ultrasound receiver respectively.

A third aspect of the invention provides a method of ultrasound imaging of internal tissue of an animal comprising:
providing an ultrasound probe having a plurality of ultrasound transducers, wherein at least one first transducer is configured to operate as an ultrasound source, and at least one second transducer is configured to operate as an ultrasound receiver;
placing the ultrasound probe on a surface of the animal;
operating a first transducer to emit an ultrasound wave and measuring a travel time of the wave directed from the first transducer via a subsurface boundary to a second transducer;
determining slowness of the wave in a plane tangent to the surface at one or both of the first and second transducer;
calculating a location of a point on the subsurface boundary on the basis of the measured travel time, and the determined slowness of the wave in the tangent plane; and,
constructing an image of the subsurface boundary utilising the calculated location of the point on the subsurface boundary.

In one embodiment of this aspect slowness in the tangent plane is determined for only one of a source domain or a receiver domain, and the method may further comprise determining curvature of the wave in a same domain as that at which the slowness in the tangent plane is determined, and wherein calculating the location of the point is further based on the curvature of the wave.

In this embodiment when the first transducer is at a location (x₁,z₁) and the second transducer is at a location (x₂,z₂) and travel time of a wave from the first transducer to the second transducer via the boundary is measured as t, the slowness of the wave in a plane tangent to the surface at the second transducer is defined as:

$${p}_{x} = \frac{d t}{d {x}_{2}} ;$$

curvature of the wave is defined as

$${p}_{xx} = \frac{{d}^{2} t}{d {x}_{2}^{2}}$$

and
the location of a point on the boundary (x,z) impinged by the wave is determined as:

$$x = \frac{2 {p}^{2} {x}_{2} ( t + \sqrt{{p}^{2} - {p}_{x}^{2}} ( {z}_{2} - {z}_{1} ) ) + {p}_{x} ( {p}^{2} ( {x}_{1}^{2} - {x}_{2}^{2} + {( {z}_{2} - {z}_{1} )}^{2} ) - {t}^{2} )}{2 {p}^{2} ( t + {p}_{x} ( {x}_{1} - {x}_{2} ) + \sqrt{{p}^{2} - {p}_{z}^{2}} ( {z}_{2} - {z}_{1} ) )}$$

$$and$$

$$z = \frac{\sqrt{{p}^{2} - {p}_{x}^{2}} ( {t}^{2} - {p}^{2} ( {( {x}_{2} - {x}_{1} )}^{2} + {z}_{1}^{2} - {z}_{2}^{2} ) ) + 2 {p}^{2} {z}_{2} ( t - {p}_{x} ( {x}_{2} - {x}_{1} ) )}{2 {p}^{2} ( t + {p}_{x} ( {x}_{1} - {x}_{2} ) + \sqrt{{p}^{2} - {p}_{x}^{2}} ( {z}_{2} - {z}_{1} ) )}$$

$$where {p}^{2} = {p}_{x}^{2} + {tp}_{xx} .$$

In an alternate embodiment of this aspect slowness in the tangent plane is determined at both the first transducer and the second transducer and calculation of the location of the point is based on the determined slowness at both the first and second transducers.

The alternate embodiment when:
the first transducer is at a location (xₛ,0);
the second transducer is at a location (xᵣ,0);
travel time of a wave from the first transducer to the second transducer via a point (x,z) on the boundary is measured as t;
V=1/p is velocity through the internal tissue; and,
z is the vertical depth of the reflection point (x,z):
then:
horizontal slowness at the first transducer is pᵣ where:

$${p}_{r} ( x , t ) = \frac{d t}{d {x}_{r}} = \frac{{x}_{r} - x}{{V}^{2} \sqrt{{z}^{2} + {( {x}_{r} - {x}_{m} )}^{2} / {V}^{2}}}$$

horizontal slowness at the second transducer is denoted by pₛ:

$${p}_{s} ( x , t ) = \frac{d t}{d {x}_{s}} = \frac{{x}_{s} - x}{{V}^{2} \sqrt{{z}^{2} + {( {x}_{s} - x )}^{2} / {V}^{2}}}$$

$$x = {x}_{s} - {dp}_{s} \frac{t - {dp}_{r}}{t ( {p}_{r} - {p}_{s} ) + 2 {dp}_{s} {p}_{r}}$$

$$z = \frac{\vert {x}_{s} - x \vert}{V} \frac{\sqrt{1 / {V}^{2} - {p}_{s}^{2}}}{\vert {p}_{s} \vert}$$

$$and$$

$${V}^{2} = \frac{{x}_{s} - x}{{tp}_{s}} + \frac{{x}_{r} - x}{{tp}_{r}}$$

$$where d = {x}_{r} - {x}_{s}$$

A fourth aspect of the invention there is provided a method of operating an ultrasound imaging device having an ultrasound probe provided with a plurality of ultrasound sources and receivers, a processor for processing data received by the receivers, and an imaging device on which an image of the subsurface of a body can be displayed or recorded, the method comprising:
measuring travel time of respective ultrasound waves directed from one or more of the sources via one or more subsurface boundaries to one or more of the receivers;
determining slowness of the waves in a plane tangent to the surface of the body, and curvature of the waves;
calculating the location of the respective subsurface boundaries on the basis of the measured travel time, the slowness of the waves in the tangent plane, and curvature of the respective waves; and,
constructing an image of the subsurface utilising the measured travel times, and the determined slowness and curvature.

The image may be constructed in real time or in near real time.

The slowness of the waves in a plane tangent to the surface of the body, and curvature of the waves may be determined in a selected one of a source domain or receiver domain only and the slowness and wave curvature are determined as first and second derivatives of travel time with respect to the selected domain.

In an alternate embodiment slowness of the wave in a plane tangent to the surface of the body is determined in both a source domain and a receiver domain and the slowness of the waves is determined as a first derivative of travel time at both a source and a receiver.

### BRIEF DESCRIPTION OF THE DRAWINGS

An embodiment of the present invention will now be described by way of example only with reference to the accompanying drawings in which:

FIG. 1 is a block diagram depicting an embodiment of a method of determining a location of a subsurface boundary;

FIG. 2 illustrates a spatial relationship between a wave source, a wave receiver and reflection point on a subsurface boundary of a body being imaged by the method;

FIG. 3 illustrates a method of calculating horizontal slowness of a wave at a receiver;

FIG. 4 is a representation of a velocity model used for the purposes of testing an embodiment of the present method;

FIG. 5 is the representation of an image of a subsurface including subsurface boundaries produced by application of an embodiment of the present method; and,

FIG. 6 illustrates a second embodiment of a method of determining a location of a subsurface boundary.

### DETAILED DESCRIPTION OF PREFERRED EMBODIMENT

In broad terms, an embodiment of the present invention facilitates the imaging of a subsurface by utilising travel times of elastic waves through the subsurface and slowness, in a plane tangent to the surface, of a wave directed to the surface via a subsurface boundary. A subsurface boundary may exist for example as a result of a change in physical properties in the body. For example, in a seismic surveying application, the boundary may be indicative of a change in geology from for example limestone to coal. In a medical application, the boundary may arise for example due to a change from muscle tissue to cancerous tissue. The wave can be directed from a wave source to a receiver by the boundary by way of reflection or diffraction. For simplicity, and ease of description, it is assumed that a surface of the body on which an embodiment of the present method is performed is locally flat and lies in a horizontal plane and thus the slowness in the plane tangent to the surface can be considered as horizontal slowness. Embodiments of the present invention enable the determination of the location of a subsurface boundary by relying on horizontal slowness, which is the derivative of the travel time with respect to the location of the source or receiver. Although optionally both derivatives may be used. In addition to measured travel times of waves from a source to a receiver and horizontal slowness, embodiments of the method also rely on the calculation or determination of a curvature of the wave in the domain in question by means of the second derivative of the travel time with respect to the location of the source or receiver. There is no requirement for the working of embodiments of the invention to have prior knowledge of the velocity of the wave through the body in order to determine the location of a subsurface boundary. However as a result of application of the method, such velocity can be estimated.

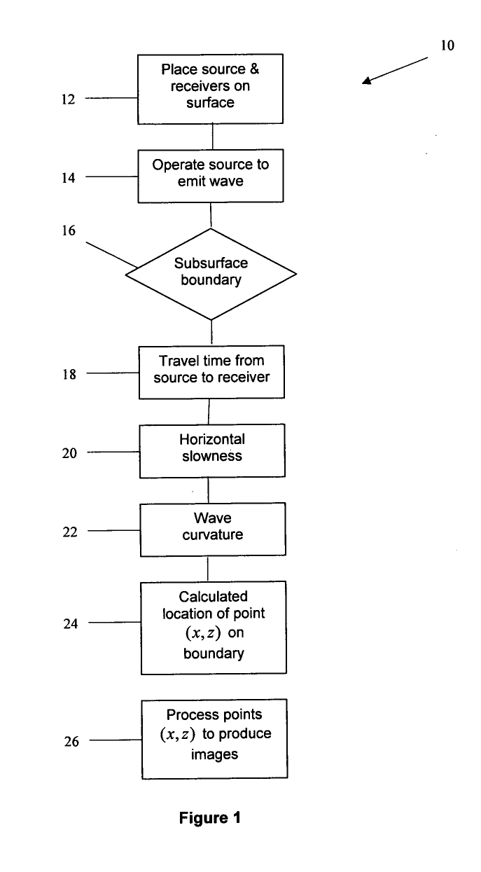

A first embodiment of a method 10 for determining the location of a subsurface boundary in the receiver domain is illustrated diagrammatically in FIG. 1. The method 10 comprises an initial step 12 of placing a wave source and one or more wave receivers on a surface of a body under investigation. At step 14, the source is operated to emit an elastic wave.

At step 16 a wave from the source impinges an internal or subsurface boundary of body and the wave is directed back to a receiver by way of reflection or diffraction. At step 18, a measure is taken of the travel time of the wave from the source to a point on the boundary and subsequently to a receiver. At step 20, a determination is made of the horizontal slowness of the travel time at the receiver. This may be determined mathematically as a derivative of the travel time with respect to the receiver location. At step 22, a determination is made of the curvature of the wave at the receiver. Mathematically this may be equated with determining the second derivative of the travel time with respect to the receiver. At step 24, with the knowledge of the travel time, the horizontal slowness, and wave curvature, a determination or calculation can be made of a point on the subsurface boundary from which the wave is reflected or diffracted to the receiver. For the sake of simplicity of description, in the remainder of the specification, reference will be made only to a wave reflecting from a point on the boundary. However application of the method and the method steps are equally adaptable to a diffracted wave.

At step 26, various images may be constructed utilising the determined location of the points on the subsurface boundary. These images may include for example a representation of wave velocities within the body or representation of the internal (subsurface) structure of the body including any subsurface boundary.

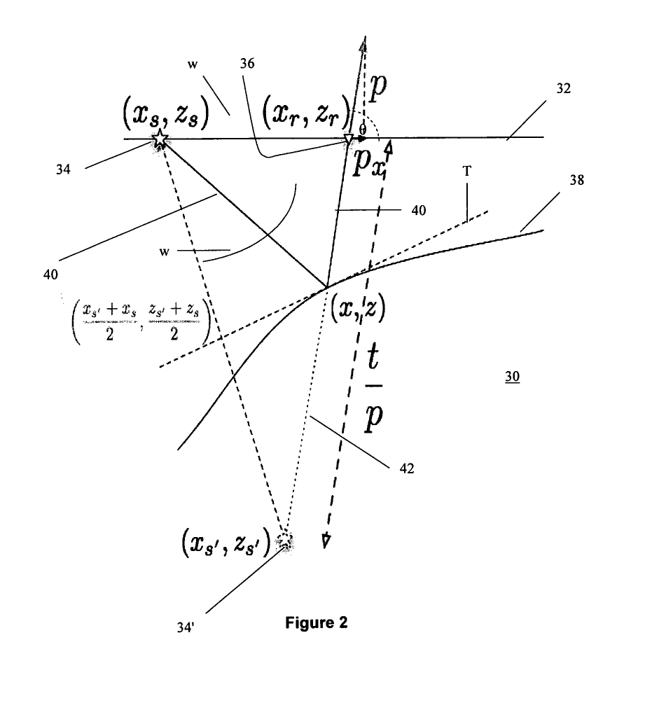

FIG. 2 illustrates the theory employed, or embodied, by method 10. FIG. 2 illustrates a body of material 30 having a surface 32. A source 34 is positioned at a location (xₛ,zₛ). A receiver 36 is disposed at a location (xᵣ,zᵣ) on the surface 32. Line 36 depicts a subsurface boundary within the body 30. A wave w is emitted from the source 32 striking boundary 36 at a location (x,z) and is reflected to the receiver 36. Line 40 depicts the normal of a wave front of wave w propagating from source 34 to point (x,z) and to receiver 36. The travel time of the wave w from source 34 to point (x,z) and subsequently to the receiver 36 is denoted as t and it is assumed that the wave propagates through the body 30 from source 34 to receiver 36 via the boundary 38 at a uniform velocity v. The slowness p of the wave w is the reciprocal of velocity, i.e.

$$p = \frac{1}{v} .$$

From the view point of the receiver 36, the received reflected wave has the appearance of a wave emitted from an image of the source 34′ at location (x_{s′},z_{s′}). This point is the reflection of the source 34 about a tangent plane T to the point (x,z) on the boundary 38. The source image 34′ is spaced from the receiver 36 by the distance

$$tv = \frac{t}{p}$$

along a line 42 which is coincident with the direction of propagation of the wave from the reflection point (x,z) to the location of the receiver 36.

Thus if angle θ of line 42 relative to surface 32 can be determined then it is possible to locate the position of the source image 34′. Subsequently with knowledge of the location of the source image 34′ it is possible to determine the location of reflection point (x,z) on boundary 38.

To determine angle θ we have regard to the slowness p. Slowness p is a vector directed along a portion of line 40 from reflection point (x,z) to the receiver 36 at (xᵣ,zᵣ). Slowness p can be resolved into a component in the plane of surface 32 at the location of receiver 36, which is termed horizontal slowness and denoted as pₓ and a component in a perpendicular direction, which for this exercise is of no specific interest. The horizontal slowness is a measure of the rate of change of travel time at receiver 36 and can be expressed mathematically as the first derivative of travel time with respect to receiver location. Thus

$${p}_{x} = \frac{d t}{d {x}_{r}} .$$

The angle θ of line 42 is thus arccos

$$\frac{{p}_{x}}{p}$$

From this, it is possible to determine the coordinates (x_{s′},z_{s′}), of the image 34′ as:

$${z}_{{s}^{′}} = {z}_{r} + \frac{t}{p} \sqrt{1 - {( \frac{{p}_{x}}{p} )}^{2}} = {z}_{r} + \frac{t}{{p}^{2}} \sqrt{{p}^{2} - {p}_{z}^{2}}$$

$$and$$

$${x}_{{s}^{′}} = {x}_{r} = \frac{{tp}_{z}}{{p}^{2}}$$

The location of the reflection point (x,z) is at the intersection of the tangent plane T, and line 42 extending from the source image 34′ to the receiver 36. Accordingly using basic trigonometry, the coordinates of the reflection point (x,z) are given by:

$$\begin{matrix} x = \frac{{x}_{r} ( \begin{matrix}{x}_{s}^{2} - {x}_{{s}^{′}}^{2} + \\ {( {z}_{s} - {z}_{{s}^{′}} )}^{2}\end{matrix} ) + {x}_{{s}^{′}} ( \begin{matrix}{x}_{{s}^{′}}^{2} - {x}_{s}^{2} + {z}_{{s}^{′}}^{2} - {z}_{s}^{2} + \\ 2 {z}_{r} ( {z}_{s} - {z}_{{s}^{′}} )\end{matrix} )}{2 ( ( {x}_{r} - {x}_{{s}^{′}} ) ( {x}_{s} - {x}_{{s}^{′}} ) + ( {z}_{r} - {z}_{{s}^{′}} ) ( {z}_{s} - {z}_{{s}^{′}} ) )} \\ and & ( 1 ) \\ z = \frac{2 ( \begin{matrix}{x}_{r} {x}_{s} {z}_{{s}^{′}} - {x}_{s} {x}_{{s}^{′}} {z}_{r} - \\ {x}_{r} {x}_{{s}^{′}} {z}_{{s}^{′}}\end{matrix} ) + {x}_{{s}^{′}}^{2} ( {z}_{r} + {z}_{{s}^{′}} ) + ( {z}_{r} - {z}_{{s}^{′}} ) ( \begin{matrix}{x}_{s}^{2} + {z}_{s}^{2} - \\ {z}_{{s}^{′}}^{2}\end{matrix} )}{2 ( ( {x}_{r} - {x}_{{s}^{′}} ) ( {x}_{s} - {x}_{{s}^{′}} ) + ( {z}_{r} - {z}_{{s}^{′}} ) ( {z}_{s} - {z}_{{s}^{′}} ) )} & ( 2 )\end{matrix}$$

After substituting for x_{s′} and z_{s′} we get the coordinates of the reflector, namely,

$$\begin{matrix}x = \frac{2 {p}^{2} {x}_{2} ( t + \sqrt{{p}^{2} - {p}_{x}^{2}} ( {z}_{2} - {z}_{1} ) ) + {p}_{x} ( {p}^{2} ( \begin{matrix}{x}_{1}^{2} - {x}_{2}^{2} + \\ {( {z}_{2} - {z}_{1} )}^{2}\end{matrix} ) - {t}^{2} )}{2 {p}^{2} ( t + {p}_{x} ( {x}_{1} - {x}_{2} ) + \sqrt{{p}^{2} - {p}_{z}^{2}} ( {z}_{2} - {z}_{1} ) )} \\ and & ( 3 ) \\ z = \frac{\sqrt{{p}^{2} - {p}_{x}^{2}} ( {t}^{2} - {p}^{2} ( \begin{matrix}{( {x}_{r} - {x}_{s} )}^{2} + \\ {z}_{s}^{2} - {z}_{r}^{2}\end{matrix} ) ) + 2 {p}^{2} {z}_{r} ( t - {p}_{x} ( {x}_{r} - {x}_{s} ) )}{2 {p}^{2} ( t + {p}_{x} ( {x}_{s} - {x}_{r} ) + \sqrt{{p}^{2} - {p}_{x}^{2}} ( {z}_{r} - {z}_{s} ) )} & ( 4 )\end{matrix}$$

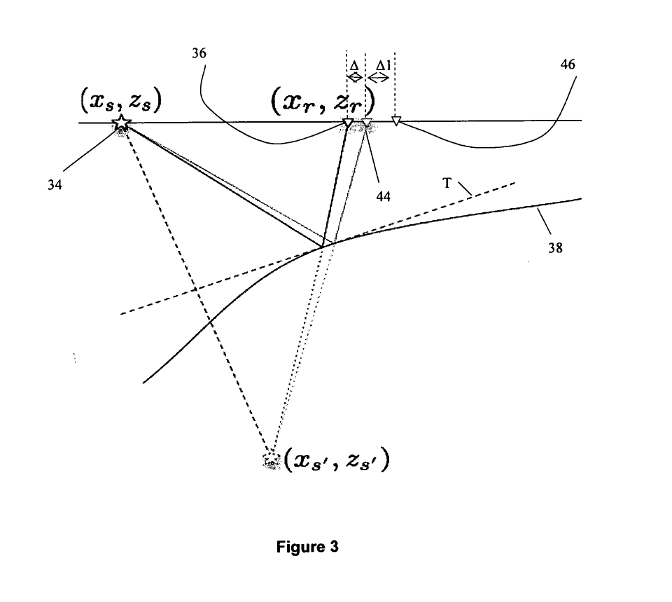

To use these expressions, we need to know the slowness, p, of the body 30. We can find this slowness if we assume that the reflector can be approximated locally by a plane. In such a case, the location of the image of the source does not change if we infinitesimally change the location of the receiver, as illustrated in FIG. 3. In other words, the derivative of x_{s′} and z_{s′} with respect to xᵣ must be zero:

$$0 = 1 - {( \frac{{p}_{z}}{p} )}^{2} - \frac{t}{{p}^{2}} {p}_{xx} , where$$

$${p}_{xx} := \frac{d {p}_{x}}{d {x}_{r}} = \frac{{d}^{2} t}{d {x}_{r}^{2}}$$

Solving this equation for p gives:

$$\frac{1}{{v}^{2}} ≡ {p}^{2} = {p}_{x}^{2} + {tp}_{xx} \qquad ( 5 )$$

To obtain the expressions for constant velocity depth migration, we substitute expression p² to expressions for x and z.

In a practical sense, the horizontal slowness pₓ can be determined also by positioning a second receiver 44 at a known spatial relationship (i.e. distance) from the receiver 36. In FIG. 3, a second receiver 44 is located at the coordinates (xᵣ+Δ,zᵣ). Thus the distance between the first receiver 36 and second receiver 44 is A. If the travel time of a wave w from source 34 to receiver 36 is time t₁, and travel time of the wave from source 34 to receiver 44 is t₂, then in effect the horizontal slowness

$${p}_{x} = \frac{{t}_{2} - {t}_{1}}{Δ} .$$

This approximates to the first derivative of travel time with respect to the receiver. Thus, in a physical system, by measuring the travel times of the wave from the source to the receivers, and knowing the spacing Δ between the receivers, it is possible to determine horizontal slowness pₓ. By similar process, by incorporating a third receiver 46 at a known spacing Δ₁ from the second receiver 44, it is possible to determine the rate of change of horizontal slowness pₓ which is denoted as

$${p}_{xx} = \frac{{t}_{3} - 2 {t}_{2} + {t}_{1}}{Δ . {Δ}_{1}}$$

where t₃ is travel time from source 34 to the third receiver 46.

This expression is also equivalent to calculating the curvature of the wave w at the receiver.

Accordingly, by recording or measuring wave travel times, and knowing the spatial relationship between source and receivers, it is possible to calculate slowness p, and the coordinates (x,z) on boundary 38.

The above process is described in relation to only one reflection point (x,z) on boundary 38. However in a practical system, receivers will receive the waves reflected from multiple points along boundary 38. By calculating the location of the reflection points, the location of the boundary 38 can be mapped and thus an image of the subsurface 30 may be imaged.

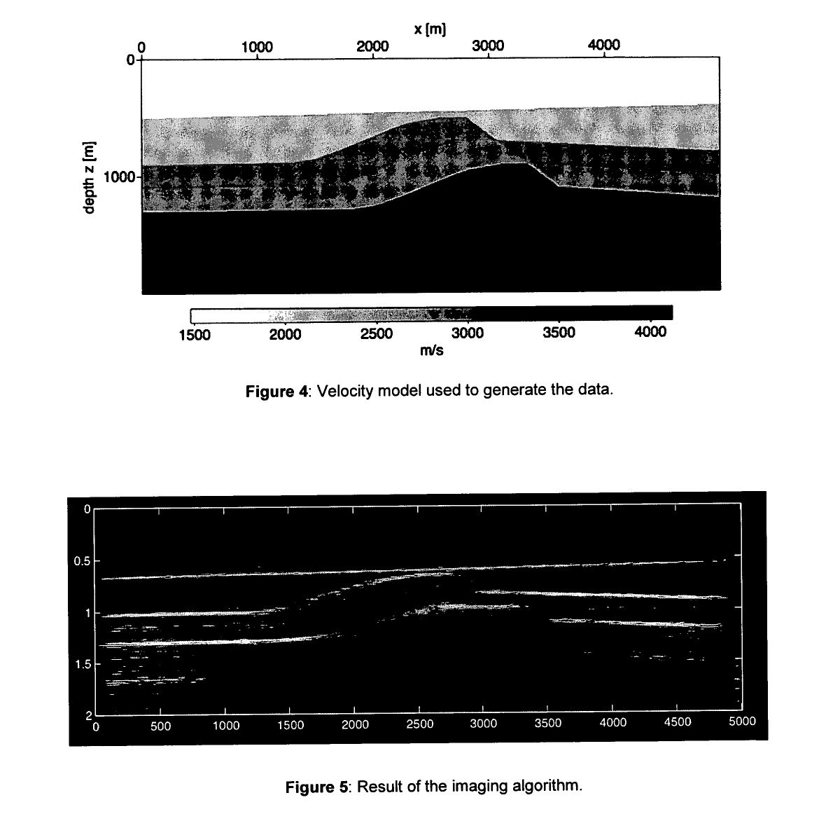

FIGS. 4 and 5 depict an example of an application of method 10 in the area of seismic exploration. A model of a subsurface was produced in accordance with FIG. 4 where velocity distribution of the subsurface is represented in a grey scale. In terms of the explanation provided above in relation to FIGS. 2 and 3, regions of the subsurface having different velocity distribution denote or are equated with one or more boundaries 38. By using the method 10 on the model shown in FIG. 4, the locations of the boundaries between regions of different velocity can be determined and imaged as shown in FIG. 5. This figure shows that the proposed method is able to clearly image reflectors (i.e. boundaries).

Embodiments of the method may be in the form of software stored on a readable medium that is accessible by the processor or computer. Embodiment of the method may further comprise a computer or other device which comprises a computer or process where the computer or processor is programmed to implement the method.

The method is readily adaptable to medical imaging and in particular ultrasound imaging. In particular the method may be employed without any physical change to existing ultrasound imaging equipment, the only change being in the manner in which data is processed by an onboard processor or computer. A typical ultrasound probe comprises a plurality of transducers in a fixed linear (i.e. two dimensional) or three dimensional array. Each transducer is able to act selectively as a source or a receiver. Thus the probe may be programmed or driven to provide for example a single source and three or more receivers. By measuring travel times of ultrasound waves emitted from the source and received by the receivers, and by knowing the spatial relationship between the source and the receivers, reflection points along internal boundaries can be located and these points processed to produce real time or at least near real time images for diagnostic and/or therapeutic purposes.

As explained previously, the present method also enables calculation of wave slowness (or velocity) through the body. The slowness will be dependent upon tissue type. As a consequence, use of the present method also facilitates diagnosis by correlating velocity with known tissue type. It is believed that application of this method in the medical ultrasound imaging area will result in sharper clearer representations of internal tissue and can be subsequently used to focus or steer an ultrasound beam for therapeutic purposes. For example this may be used to accurately locate gallstones, and then enable ultrasonic treatment of the gallstones by enabling a tighter and better focussed ultrasound beam for the purposes of extracorporeal shock wave lithotripsy.

In the above described embodiments, it is assumed that the source and receivers lie in a plane. However with many ultrasound probes, ultrasonic transducers are located about a curved head or surface. The method is equally applicable irrespective of the relative spatial relationship between the source and receivers provided only that the spatial relationship is known. While the mathematics will be different arise from the curvilinear spatial relationship between source and receivers the method steps of measuring travel time, horizontal slowness and wave curvature (or travel time, and the first and second derivatives of travel time with respect to receiver location) remain the same.

In the above embodiment where horizontal slowness and wave curvature are determined at the receiver the method may be described as being applied in the receiver domain. However as will be apparent to those of ordinary skill in the art, the method may be equally applied in the source domain where horizontal slowness and wave curvature are determined at the source. For example, by having three sources and one receiver all having known spatial relationship, the same method is used however the method is now in the source domain rather than the receiver domain. In this regard, the horizontal slowness is the slowness at a source and determined as the difference in travel time from a first source and second source to the same receiver divided by the spacing between the sources, equating to the first derivative of travel time with respect to the source. Wave front curvature is similarly determined as the rate of change of horizontal slowness with respect to the source.

In terms of equations (1)-(5) when the source domain is used these equations are simply re-written by swapping the co-ordinates xₛ,zₛ with xᵣ,zᵣ respectively. Thus the above method can be termed as a one domain tow derivative method as wave properties are determined at one domain (e.g. source or receiver) and the properties include the first and second derivatives of travel time with respect to the domain in question. Accordingly in a general sense if a first domain device is at a location (x1,z₂) and a second domain device is at a location (x₂,z₂) where the first domain device is one of a wave source and a wave receiver, and the second domain device is the other or the source and receiver then equations (4) and (5) which provide the coordinates of the reflection point (x,z) on boundary 38 may be re-written as:

$$\begin{matrix}x = \frac{2 {p}^{2} {x}_{2} ( t + \sqrt{{p}^{2} - {p}_{x}^{2}} ( {z}_{2} - {z}_{1} ) ) + {p}_{x} ( {p}^{2} ( \begin{matrix}{x}_{1}^{2} - {x}_{2}^{2} + \\ {( {z}_{2} - {z}_{1} )}^{2}\end{matrix} ) - {t}^{2} )}{2 {p}^{2} ( t + {p}_{x} ( {x}_{1} - {x}_{2} ) + \sqrt{{p}^{2} - {p}_{z}^{2}} ( {z}_{2} - {z}_{1} ) )} \\ and & ( 3 a ) \\ z = \frac{\sqrt{{p}^{2} - {p}_{x}^{2}} ( {t}^{2} - {p}^{2} ( \begin{matrix}{( {x}_{2} - {x}_{1} )}^{2} + \\ {z}_{1}^{2} - {z}_{2}^{2}\end{matrix} ) ) + 2 {p}^{2} {z}_{2} ( t - {p}_{x} ( {x}_{2} - {x}_{1} ) )}{2 {p}^{2} ( t + {p}_{x} ( {x}_{1} - {x}_{2} ) + \sqrt{{p}^{2} - {p}_{x}^{2}} ( {z}_{2} - {z}_{1} ) )} & ( 4 a )\end{matrix}$$

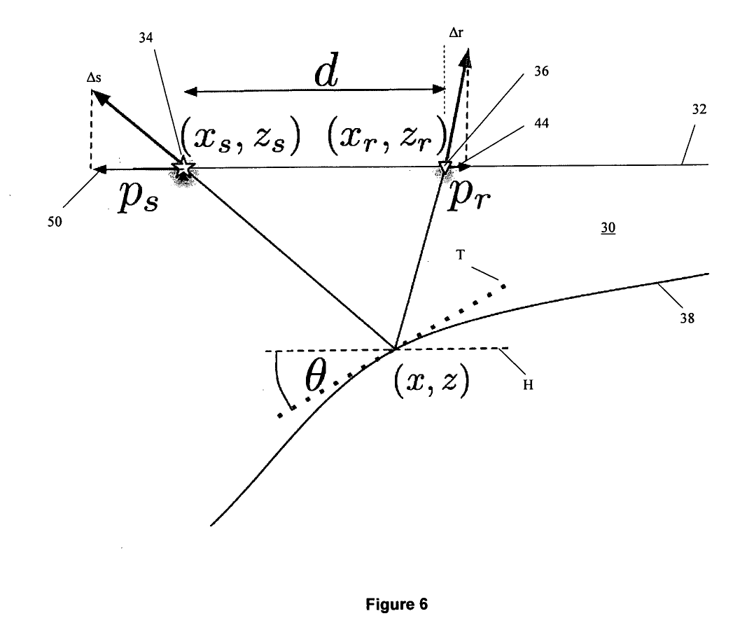

In a variation to the above described embodiment, the method may be utilised in both the source and receiver domain. Again, it is believed that this is readily achievable in the area of medical ultrasound imaging where an ultrasound probe can be configured so that for example two or more transducers are operated as sources, and two or more are operated as receivers. This method utilises horizontal slowness in both the source and receiver domains, and its underlying principles are shown in FIG. 6.

FIG. 6 illustrates the same general set up as shown in FIGS. 2 and 3 where a source 34 and receiver 36 are placed on surface 32 of a body 30. The body 30 includes a boundary 38 denoting a line or area of demarcation of areas of the body 30 having different physical properties. Source 34 is at location (xₛ,zₛ), receiver 36 is at location (xᵣ,zᵣ) and a reflection point on the boundary 38 is denoted as location (x,z). In this embodiment θ is denoted as the angle between tangent plane T (being a plane tangent to point (x,z) on the boundary 38) and a plane H which is parallel to a line joining source 34 and receiver 36. The horizontal slowness of a wave at receiver 36 is denoted as pᵣ, while horizontal slowness of the wave at the receiver 34 is denoted as pₛ. The travel time t wave propagating between source 34 and 36 via a reflection at point (x,z) in an homogenous medium can be calculated as:

t=p\sqrt{(zᵣ−z)²+(xᵣ−x)²}+p\sqrt{(zₛ−z)²+(xₛ−x)} (6)

In this equation V=1/p is velocity through the medium, z is the vertical depth of the reflection point (x,z) and x, xₛ and xᵣ are the horizontal coordinates of the reflection point, source location and receiver location. For simplicity, we assume that zᵣ=zₛ=0. For all (x,t) points in a source or receiver gather t, xₛ and xᵣ are known and V, z (or equivalently vertical timeline) and x are unknowns.

The horizontal slowness at the source may be derived as a partial derivative of equation 6 above with respect to xᵣ and is denoted by pᵣ where:

$${p}_{r} ( x , t ) = \frac{d t}{d {x}_{r}} = \frac{{x}_{r} - x}{{V}^{2} \sqrt{{z}^{2} + {( {x}_{r} - {x}_{m} )}^{2} / {V}^{2}}} \qquad ( 7 )$$

and the partial derivative of equation 6 with respect to xₛ is denoted by pₛ:

$${p}_{s} ( x , t ) = \frac{d t}{d {x}_{s}} = \frac{{x}_{s} - x}{{V}^{2} \sqrt{{z}^{2} + {( {x}_{s} - x )}^{2} / {V}^{2}}} \qquad ( 8 )$$

In a similar fashion to that described in relation to the first embodiment, the horizontal slowness pₛ and pᵣ at the source and receiver can be measured for every sample in source and receiver gathers respectively. This requires the provision of at least a second source 50 spaced by distance Δₛ from source 34, and a second receiver 44 spaced a distance Δᵣ from source 36. In this regard, if travel time of a wave from source 34 to second receiver 44 is denoted as time t₂, and travel time from source 50 to receiver 36 is denoted as time t, then the horizontal slowness at the source pₛ may be approximated as

$$\frac{{t}_{3} - {t}_{1}}{{Δ}_{s}} ,$$

while horizontal slowness pᵣ at source 34 may be approximated as

$$\frac{{t}_{2} - {t}_{1}}{{Δ}_{r}} .$$

As the spatial relationship between the source and the receivers is known, and the respective travel times of signals between the sources and the receivers is measured, that is pₛ, pᵣ, and t are known or can be measured/approximated, then the three unknown variables in equation (6)-(8) are x, V, and z. The solution to these variables is given by:

$$\begin{matrix}x = {x}_{s} - {dp}_{s} \frac{t - {dp}_{r}}{t ( {p}_{r} - {p}_{s} ) + 2 {dp}_{s} {p}_{r}} & ( 9 ) \\ {V}^{2} = \frac{{x}_{s} - x}{{tp}_{s}} + \frac{{x}_{r} - x}{{tp}_{r}} & ( 10 ) \\ z = \frac{\vert {x}_{s} - x \vert}{V} \frac{\sqrt{1 / {V}^{2} - {p}_{s}^{2}}}{\vert {p}_{s} \vert} & ( 11 )\end{matrix}$$

In FIG. 6, the angle θ is calculated as: θ=½(arccos(pᵣV)−arccos(pₛV))

In the above, d=xᵣ−xₛ which corresponds to the source receiver offset for source 34 and receiver 36. Thus, by implementation of the above method it is possible to derive the location of the reflection point (x,z). Thus this method requires inputs pₛ and pᵣ for each sample of every trace (i.e. wave); and ideally a source at every receiver location and a receiver at every source location (herein after referred to as “reciprocal source−receiver geometry”). In the medical ultrasound application, this is readily achievable as standard ultrasound probes comprise an array of transducers which may operate either as a source or receiver.

It will be appreciated that in the application of this method, multiple reflections will be acquired from different sources at multiple points along boundary 38. In order to correctly migrate the reflection points, an appropriate work flow to derive an image of boundary 38 may be as follows:
(1) sort data by common source and subsequently calculate and save horizontal slowness at the source pₛ for all points in this gather;
(2) sort data by common receiver and calculate and save horizontal slowness at the receiver pᵣ for all points in the receiver gather;
(3) for each sample on each propagated wave, using the measured travel time t and the previously derived pₛ and pᵣ, to solve equations 9, 10 and 11 to obtain x, z, and V.

We now have a data set of migrated reflection points (x,z). which may be processed to produce a representation of the location of subsurface boundary 38. It will be understood that the method differs form that described in relation to FIGS. 2 and 3 in that two domains (source and receiver) are used and travel time and the first derivative of travel time are measured or approximated to enable determination of the location of a subsurface boundary 38.

All modifications and variations that would be obvious to persons of ordinary skill in the art deemed to be within the scope of the present invention the nature of which is to be determined from the above description and the appended claims.

1. A method of determining a location of a subsurface boundary in a body of material comprising:
providing a first domain device and second domain device on a surface of the body where the first domain device is an elastic wave source or an elastic wave receiver, and the second domain device is the other of the elastic wave source or an elastic wave receiver;
measuring travel time of a wave propagating via a subsurface boundary between the first and second domain devices;
determining: slowness of the wave at the first domain device in a plane tangent to the surface at the first domain device; and, curvature of the wave at the first domain device; and,
calculating the location of the subsurface boundary on the basis of the measured travel time, and the determined slowness of the wave and curvature of the wave.

2. The method according to claim 1 wherein determining slowness in the tangent plane comprises determining a first derivate of travel time at the first domain device.

3. e method according to claim 1 wherein determining curvature comprises determining a second derivate of travel time at the first domain device.

4. The method according to claim 1 wherein when the first domain device is at a location (x₁,z₁) and the second domain device is at a location (x₂,z₂) and travel time of a wave from the first source to the first receiver via the boundary is measured as t, the slowness of the wave in a plane tangent to the surface at the second domain device is defined as:
$${p}_{x} = \frac{d t}{d {x}_{2}} ;$$
curvature of the wave is defined as
$${p}_{xx} = \frac{{d}^{2} t}{d {x}_{2}^{2}}$$
and
the location of a point on the boundary (x,z) impinged by the wave is determined as
$$x = \frac{2 {p}^{2} {x}_{2} ( t + \sqrt{{p}^{2} - {p}_{x}^{2}} ( {z}_{2} - {z}_{1} ) ) + {p}_{x} ( {p}^{2} ( {x}_{1}^{2} - {x}_{2}^{2} + {( {z}_{2} - {z}_{1} )}^{2} ) - {t}^{2} )}{2 {p}^{2} ( t + {p}_{x} ( {x}_{1} - {x}_{2} ) + \sqrt{{p}^{2} - {p}_{z}^{2}} ( {z}_{2} - {z}_{1} ) )}$$
$$and$$
$$z = \frac{\sqrt{{p}^{2} - {p}_{x}^{2}} ( {t}^{2} - {p}^{2} ( {( {x}_{2} - {x}_{1} )}^{2} + {z}_{1}^{2} - {z}_{2}^{2} ) ) + 2 {p}^{2} {z}_{2} ( t - {p}_{x} ( {x}_{2} - {x}_{1} ) )}{2 {p}^{2} ( t + {p}_{x} ( {x}_{1} - {x}_{2} ) + \sqrt{{p}^{2} - {p}_{x}^{2}} ( {z}_{2} - {z}_{1} ) )}$$
$$where {p}^{2} = {p}_{x}^{2} + {tp}_{xx} .$$

5. The method according to claim 1 wherein providing the first and second domain devices comprises providing the first and second domain device as an ultrasound source and an ultrasound receiver respectively.

6. A method of determining a location of subsurface boundary in animal tissue comprising:
using the method according to claim 1 wherein the body of material is an animal body, and the first and second domain devices comprises an ultrasound source and an ultrasound receiver respectively.

7. A method of ultrasound imaging of internal tissue of an animal comprising:
providing an ultrasound probe having a plurality of ultrasound transducers, wherein at least one first transducer is configured to operate as an ultrasound source, and at least one second transducer is configured to operate as an ultrasound receiver;
placing the ultrasound probe on a surface of the animal;
operating a first transducer to emit an ultrasound wave and measuring a travel time of the wave directed from the first transducer via a subsurface boundary to a second transducer;
determining slowness of the wave in a plane tangent to the surface at one or both of the first and second transducer;
calculating a location of a point on the subsurface boundary on the basis of the measured travel time, and the determined slowness of the wave in the tangent plane; and,
constructing an image of the subsurface boundary utilising the calculated location of the point on the subsurface boundary.

8. The method according to claim 7 wherein slowness in the tangent plane is determined for only one of a source domain or a receiver domain, and further comprising determining curvature of the wave in a same domain as that at which the slowness in the tangent plane is determined, and wherein calculating the location of the point is further based on the curvature of the wave.

9. The method according to claim 8 wherein when the first transducer is at a location (x₁,z₁) and the second transducer is at a location (x₂,z₂) and travel time of a wave from the first transducer to the second transducer via the boundary is measured as t, the slowness of the wave in a plane tangent to the surface at the second transducer is defined as:
$${p}_{x} = \frac{d t}{d {x}_{2}} ;$$
curvature of the wave is defined as
$${p}_{xx} = \frac{{d}^{2} t}{d {x}_{2}^{2}}$$
and
the location of a point on the boundary (x,z) impinged by the wave is determined as:
$$x = \frac{2 {p}^{2} {x}_{2} ( t + \sqrt{{p}^{2} - {p}_{x}^{2}} ( {z}_{2} - {z}_{1} ) ) + {p}_{x} ( {p}^{2} ( {x}_{1}^{2} - {x}_{2}^{2} + {( {z}_{2} - {z}_{1} )}^{2} ) - {t}^{2} )}{2 {p}^{2} ( t + {p}_{x} ( {x}_{1} - {x}_{2} ) + \sqrt{{p}^{2} - {p}_{z}^{2}} ( {z}_{2} - {z}_{1} ) )}$$
$$and$$
$$z = \frac{\sqrt{{p}^{2} - {p}_{x}^{2}} ( {t}^{2} - {p}^{2} ( {( {x}_{2} - {x}_{1} )}^{2} + {z}_{1}^{2} - {z}_{2}^{2} ) ) + 2 {p}^{2} {z}_{2} ( t - {p}_{x} ( {x}_{2} - {x}_{1} ) )}{2 {p}^{2} ( t + {p}_{x} ( {x}_{1} - {x}_{2} ) + \sqrt{{p}^{2} - {p}_{x}^{2}} ( {z}_{2} - {z}_{1} ) )}$$
$$where {p}^{2} = {p}_{x}^{2} + {tp}_{xx} .$$

10. The method according to claim 8 wherein slowness in the tangent plane is determined at both the first transducer and the second transducer and calculation of the location of the point is based on the determined slowness at both the first and second transducers.

11. The method according to claim 10 wherein when:
the first transducer is at a location (xₛ,0);
the second transducer is at a location (xᵣ,0);
travel time of a wave from the first transducer to the second transducer via a point (x,z) on the boundary is measured as t;
V=1/p is velocity through the internal tissue; and,
z is the vertical depth of the reflection point (x,z):
then:
horizontal slowness at the first transducer is pᵣ where:
$${p}_{r} ( x , t ) = \frac{d t}{d {x}_{r}} = \frac{{x}_{r} - x}{{V}^{2} \sqrt{{z}^{2} + {( {x}_{r} - {x}_{m} )}^{2} / {V}^{2}}}$$
horizontal slowness at the second transducer is denoted by pₛ:
$${p}_{s} ( x , t ) = \frac{d t}{d {x}_{s}} = \frac{{x}_{s} - x}{{V}^{2} \sqrt{{z}^{2} + {( {x}_{s} - x )}^{2} / {V}^{2}}}$$
$$x = {x}_{s} - {dp}_{s} \frac{t - {dp}_{r}}{t ( {p}_{r} - {p}_{s} ) + 2 {dp}_{s} {p}_{r}}$$
$$z = \frac{\vert {x}_{s} - x \vert}{V} \frac{\sqrt{1 / {V}^{2} - {p}_{s}^{2}}}{\vert {p}_{s} \vert}$$
$$and$$
$${V}^{2} = \frac{{x}_{s} - x}{{tp}_{s}} + \frac{{x}_{r} - x}{{tp}_{r}}$$
$$where d = {x}_{r} - {x}_{s} .$$

12. A method of operating an ultrasound imaging device having an ultrasound probe provided with a plurality of ultrasound sources and receivers, a processor for processing data received by the receivers, and an imaging device on which an image of the subsurface of a body can be displayed or recorded, the method comprising:
measuring travel time of respective ultrasound waves directed from one or more of the sources via one or more subsurface boundaries to one or more of the receivers;
determining slowness of the waves in a plane tangent to the surface of the body, and curvature of the waves;
calculating the location of the respective subsurface boundaries on the basis of the measured travel time, the slowness of the waves in the tangent plane, and curvature of the respective waves; and,
constructing an image of the subsurface utilising the measured travel times, and the determined slowness and curvature.

13. The method according to claim 12 wherein the image is constructed in real time or in near real time.

14. The method according to claim 12 wherein slowness of the waves in a plane tangent to the surface of the body, and curvature of the waves is determined in a selected one of a source domain or receiver domain only and the slowness and wave curvature are determined as first and second derivatives of travel time with respect to the selected domain.

15. The method according to claim 12 wherein slowness of the waves in a plane tangent to the surface of the body is determined in both a source domain and a receiver domain and the slowness of the waves is determined as a first derivative of travel time at both a source and a receiver.

16. The method according to claim 14 wherein determining the slowness, wave curvature and location of a subsurface boundary is in accordance with claim 4.

17. The method according to claim 15 wherein when:
the first transducer is at a location (xₛ,0);
the second transducer is at a location (xᵣ,0);
travel time of a wave from the first transducer to the second transducer via a point (x,z)
on the boundary is measured as t;
V=1/p is velocity through the internal tissue; and,
z is the vertical depth of the reflection point (x,z):
then:
horizontal slowness at the first transducer is pᵣ where:
$${p}_{r} ( x , t ) = \frac{d t}{d {x}_{r}} = \frac{{x}_{r} - x}{{V}^{2} \sqrt{{z}^{2} + {( {x}_{r} - {x}_{m} )}^{2} / {V}^{2}}}$$
horizontal slowness at the second transducer is denoted by pₛ:
$${p}_{s} ( x , t ) = \frac{d t}{d {x}_{s}} = \frac{{x}_{s} - x}{{V}^{2} \sqrt{{z}^{2} + {( {x}_{s} - x )}^{2} / {V}^{2}}}$$
$$x = {x}_{s} - {dp}_{s} \frac{t - {dp}_{r}}{t ( {p}_{r} - {p}_{s} ) + 2 {dp}_{s} {p}_{r}}$$
$$z = \frac{\vert {x}_{s} - x \vert}{V} \frac{\sqrt{1 / {V}^{2} - {p}_{s}^{2}}}{\vert {p}_{s} \vert}$$
$$and$$
$${V}^{2} = \frac{{x}_{s} - x}{{tp}_{s}} + \frac{{x}_{r} - x}{{tp}_{r}}$$
$$where d = {x}_{r} - {x}_{s} .$$
